(19) United States
(12) Patent Application Publication
(10) Pub. No.: US 2025/0387154 A1
(43) Pub. Date: Dec. 25, 2025

(54) ACCESS DEVICE WITH AN ATRAUMATIC TIP AND METHODS AND SYSTEMS FOR CREATION THEREOF

(71) Applicant: Circa Scientific, Inc., Englewood, CO (US)

(72) Inventors: Christopher Hasselwander, Carlsbad, CA (US); Matthew Spencer Toone, Sandy, UT (US)

(73) Assignee: Circa Scientific, Inc., Englewood, CO (US)

(21) Appl. No.: 19/243,647

(22) Filed: Jun. 19, 2025

Related U.S. Application Data
(60) Provisional application No. 63/661,888, filed on Jun. 19, 2024.

(51) Int. Cl. A61B 18/14; A61B 17/00; A61B 17/34; A61B 18/00

(52) U.S. Cl. CPC A61B 18/14; A61B 2017/00526; A61B 2017/3454; A61B 2018/00077; A61B 2018/00083; A61B 2018/00107

(57) ABSTRACT

Provided is a method for creating an atraumatic tip using a laser, the method comprising positioning a puncture device distal end relative to the laser, wherein the device is configured to rotate around a longitudinal axis to intersect the laser beam; directing the laser beam to the distal end to create a desired shape of the distal end of the body defining a first tip profile; directing the laser beam towards the first tip profile to create a fillet on the distal end of the body, wherein creating the fillet may comprise at least one tip profile, wherein each tip profile comprises a diameter ablation profile; and directing the laser beam to the fillet to smooth the distal end of the body, wherein the smoothing of the distal end of the body comprises at least one tip profile comprising a diameter and a power level.

### CROSS-REFERENCE TO RELATED APPLICATIONS

This application claims priority to U.S. Application No. 63/661,888, titled ACCESS DEVICE WITH AN ATRAUMATIC TIP AND METHODS AND SYSTEMS FOR CREATION THEREOF, filed Jun. 19, 2024, which is hereby incorporated by reference in its entirety.

### FIELD OF INVENTION

The present technology relates generally to medical devices, systems, and associated methods. More particularly, the present technology relates to devices, methods, and systems for an access device with an atraumatic tip and fabrication thereof.

### BACKGROUND

The use of atraumatic tips is particularly useful in medical applications, such as surgery, where the instruments are often introduced in small regions and require precision to avoid making undesired incisions or punctures to the surrounding area. For example, transseptal crossing devices, commonly used in cardiac surgical applications, are often passed through a vein into a chamber of the heart, where a puncture to the cardiac wall is made. When advancing the crossing device through the vein and into position to create the puncture, care must be taken to avoid causing damage to the veins or other structures when positioning the crossing device in the desired position.

As a result, some have proposed alternative methods of puncturing the cardiac wall, or other structures, to reduce inadvertent damage during use. Indeed, one proposed method of puncturing the cardiac wall includes using an atraumatic tip through the application of RF energy to a region desired to be punctured. While these proposals reduce the potential damage caused by the puncture means of the crossing device, there remains a need for reducing damage caused by other portions of the crossing device.

The need for an atraumatic tip is not limited to the tip itself, but is also needed in the surrounding area, where layers of materials may be applied to give the device a desired effect, such as insultation and electrical conductivity. This may result in a rough or uneven surface at or near the atraumatic tip.

Rough surfaces can create undesired tearing or other damage to the target area, which can impact healing and can create surgical complications. Further, rough edges can catch and tear undesired regions when the crossing device, or other device, is being advanced to the desired region, which can create damage despite the use of an atraumatic tip.

Thus, there is a need for an improved device with an atraumatic tip and method for making said atraumatic tips with smooth transitions between the tip and surrounding surfaces, particularly for medical devices used in transseptal procedures where tissue damage must be minimized.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for creating an atraumatic tip on a tubular device is provided. The method includes positioning a tubular device relative to a laser, the tubular device comprising a body and a distal end. The method further includes directing a laser beam to the distal end of the body in a shaping step to create a shaped tip, the shaping step comprising a first number of passes with a first ablation profile diameter. The method also includes directing the laser beam towards the shaped tip in a filleting step to create a filleted tip, the filleting step comprising a second number of passes with a second ablation profile diameter, wherein the second ablation profile diameter is larger than the first ablation profile diameter. Additionally, the method includes directing the laser beam to the filleted tip in a smoothing step to create a smoothed tip, the smoothing step comprising a third number of passes with a third ablation profile diameter, wherein the third ablation profile diameter is larger than the second ablation profile diameter. The smoothed tip comprises a first smoothed section positioned substantially perpendicular to a longitudinal axis of the tubular device and a second smoothed section positioned at a non-orthogonal angle relative to the longitudinal axis, and wherein the first smoothing section comprises between 20% to 50% of a distal end face of the tubular device.

According to other aspects of the present disclosure, the method may include one or more of the following features. The first ablation profile diameter may be approximately 30 μm. The second ablation profile diameter may be approximately 200 μm. The third ablation profile diameter may be approximately 300 μm. The first number of passes may be greater than the second number of passes. The second number of passes may be greater than the third number of passes. The filleting step may further comprise a fourth number of passes with a fourth ablation profile diameter, wherein the fourth ablation profile diameter is smaller than the second ablation profile diameter. The fourth ablation profile diameter may be approximately 100 μm. The filleting step may further comprise a fifth number of passes with a fifth ablation profile diameter, wherein the fifth ablation profile diameter is smaller than the fourth ablation profile diameter. The fifth ablation profile diameter may be approximately 50 μm. The smoothing step may be performed at a lower power level than the shaping step and the filleting step. The power level of the smoothing step may be approximately 75% of the power level used in the shaping step and the filleting step. The tubular device may comprise a coating, and wherein the smoothing step ablates approximately half of the coating thickness at the distal end of the body. The non-orthogonal angle of the second smoothed section relative to the first smoothed section may be between 100 degrees and 140 degrees. The smoothed tip may comprise smoothed inflection sections between the first smoothed section and the second smoothed section.

According to another aspect of the present disclosure, a puncture device is provided. The puncture device includes a body having a distal end, the body formed from a cannula and a coating, wherein the coating is disposed coaxially externally to the cannula. The puncture device also includes an atraumatic tip at the distal end of the body, the atraumatic tip comprising: a first smoothed section positioned generally perpendicular to a longitudinal axis of the body; a second smoothed section positioned at an obtuse angle relative to the first smoothed section; and smoothed inflection sections between the first smoothed section and the second smoothed section, wherein the first smoothed section, the second smoothed section, and the smoothed inflection sections define a frontal annular portion of the distal end.

According to other aspects of the present disclosure, the puncture device may include one or more of the following features. The first smoothed section, the second smoothed section, and the smoothed inflection sections may each incorporate both the cannula and the coating. The cannula may be electrically conductive and the coating may be electrically insulative. The puncture device may further comprise a radiofrequency (RF) electrical generator configured to deliver RF energy to the distal tip. The obtuse angle may be between 100 and 140 degrees, and the first smoothed section may account for 20-40 percent of the frontal annular portion of the distal end.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

The patent or application file contains at least one drawing executed in color. Copies of this patent or patent application publication with color drawing(s) will be provided by the Office upon request and payment of the necessary fee.

FIG. 1 illustrates a flowchart depicting a method for creating an atraumatic tip using a laser beam, according to aspects of the present disclosure.

FIGS. 2A-2C shows multiple views of a tubular device after a shaping step of laser processing, according to an embodiment.

FIGS. 3A-3C shows multiple views of a tubular device after a filleting step of laser processing, according to aspects of the present disclosure.

FIG. 4A-4C shows multiple views of a tubular device showing details of its distal end configuration, according to aspects of the present disclosure.

FIGS. 4D-4G are microscopic views of the distal end of the tubular device following the smoothing step.

FIG. 5 illustrates a flowchart depicting an exemplary method for creating an atraumatic tip using a laser beam, according to aspects of the present disclosure.

FIG. 6A illustrates a cross-sectional view of a native coated tube with a squared-off end, showing the initial state before laser processing.

FIG. 6B illustrates the tube after the first laser pass, depicting initial material removal and the beginning of edge contouring.

FIG. 6C illustrates the result of the second laser pass, showing further material removal and more pronounced filleting.

FIG. 6D illustrates the tube following the third laser pass, illustrating continued refinement of the fillet profile with increased curvature.

FIG. 6E illustrates the outcome of the fourth laser pass, demonstrating a more defined fillet shape with smoother transitions.

FIG. 6F illustrates the tube after the fifth laser pass, showing a nearly complete fillet profile with minimal sharp edges remaining.

FIG. 6G illustrates the final smoothing pass result, presenting a finished fillet with a uniform, smooth surface transition from the tube end to the side wall.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

In one embodiment, the process may be a method for creating an atraumatic tip on a puncture device. The puncture device may be any device used for puncturing a desired region, such as a tissue crossing device, a needle, or any other device used for puncturing. The atraumatic tip may be at least partially atraumatic and may comprise a dilation portion. The method may comprise a shaping step, a filleting step, and a smoothing step, wherein each step comprises providing a laser to the puncture device. While reference is made throughout to the laser, other cutting means are contemplated.

The tubular body includes a cannula with a central lumen extending through it. This lumen may be sized to allow a guide wire to slide within it. In some implementations, it may be desirable to electrically isolate the guidewire from certain portions of the cannula. This can be achieved by incorporating a tubular insulation layer positioned between the guide wire and the inner surface of the cannula. In one embodiment, the insulation layer takes the form of a coating or tubular sleeve surrounding the guide wire. Another tubular insulation layer may also be applied to the exterior of the cannula to electrically isolate it from the patient.

The tubular body of the device (also referred to herein as the “puncture device”) may be constructed using a combination of materials to achieve optimal conductivity, structural integrity, and biocompatibility. The core of the tubular body may be composed of a highly conductive metal, such as stainless steel, nitinol, a copper alloy, or similar material. These materials may offer excellent electrical conductivity while providing the necessary column strength and flexibility. The choice between these metals may depend on the desired balance between conductivity and mechanical properties, with stainless steel offering high strength, nitinol providing superelasticity, and copper alloys potentially offering superior conductivity.

Surrounding the conductive metal core, a biocompatible polymer material jacket may be applied. This jacket may be composed of materials such as polyetheretherketone (PEEK), polyimide, or high-density polyethylene (HDPE). PEEK may offer excellent mechanical strength and chemical resistance, while polyimide provides high temperature resistance and dimensional stability. HDPE may be chosen for its low coefficient of friction and good impact resistance. The selection of the jacket material may be based on the specific requirements for flexibility, durability, and compatibility with the metal core and subsequent coatings.

In the distal region, where energy occurs, specialized conductive materials may be employed. For the energy delivery tip, materials such as platinum-iridium alloys, gold, or silver-palladium alloys may be used. These materials offer excellent conductivity and biocompatibility, with platinum-iridium providing high radiopacity for improved visibility under imaging. The exposed conductive areas may be treated or textured to enhance their surface properties, potentially improving energy transfer efficiency or reducing tissue adhesion. However, the body of the cannula and the conductive tip may be composed of the same material, where the cannula and the conductive tap may be continuous or of unibody construction. Accordingly, the cannula may be circumferentially surrounded by a coating, wherein the cannula and/or the conductive tip are conductive and the coating may be an insulator.

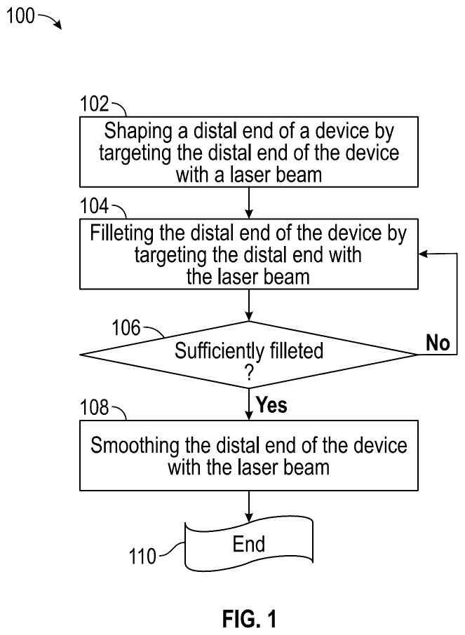

FIG. 1 illustrates one method 100 for creating the atraumatic tip of the puncture device. The puncture device may be positioned relative to the laser to permit a laser beam emitting from the laser to contact the puncture device. In the preferred setup, the laser system may employ two axes of motion to enhance precision and flexibility during processing. The object can be positioned on a rotary device to provide rotational movement, while the laser is configured to move along the Z-axis. In a first step of the method, the puncture device may be targeted with a laser beam emitted from the laser to cut a desired shape on the distal end of the puncture device. In a second step of the method, the distal end of the puncture device may be targeted by the laser beam to fillet the distal end face. It is contemplated that the step of filleting may be continued until the distal end of the puncture device has been sufficiently filleted, as discussed in more detail herewith. Once the distal end of the puncture device has been sufficiently filleted, the distal end of the puncture device may be smoothed. Smoothing may occur by passing the laser beam over the distal end of the puncture device to create a smooth surface.

The method 100 comprises several key steps that progressively shape, fillet, and smooth the distal end of the device to create an atraumatic profile.

The method begins with a shaping step 102, where a distal end of a device is targeted with a laser beam to create a desired initial shape. This initial step may involve cutting the distal end to achieve a specific profile, such as a beveled or non-orthogonal face. The result of the shaping step 102 may be referred to as the shaped tip 200. The laser parameters in this step may be configured to provide precise cutting without causing excessive thermal effects on the surrounding material. For example, the laser parameters are configured to protect the coating from thermal damage, e.g., burning, swelling, sharp edges, and the like. The selected laser frequency (femtosecond pulse lengths) may prevent overheating by applying localized heat and by ablating material before excessive heat can spread through the rest of the substrate.

Following the shaping step, the method proceeds to a filleting step 104 where the laser beam targets the distal end to create a fillet. This step may involve multiple passes of the laser beam, each with a specific ablation profile diameter and power setting. The filleting process aims to round off any sharp edges created during the initial shaping step, gradually transitioning the geometry towards a more atraumatic profile.

The method then includes a decision point 106 to determine if the distal end has been sufficiently filleted. This step allows for iterative refinement of the fillet, ensuring that the desired level of smoothness and curvature is achieved. If the distal end has not been sufficiently filleted, the method returns to the filleting step 104 for additional passes. The confirmation of whether the filleting step 104 requires additional passes may be determined by visual inspection. Such a visual inspection may be conducted with use of a jig and/or calipers, computer aided determination (for example, with the use of CNC or the like), or other tolerance checks. The result of the filleting step 104 (or the decision point 106, if the distal end was not sufficiently filleted initially) may be referred to as the filleted tip 300.

Once the distal end has been sufficiently filleted, the method proceeds to a smoothing step 108. In this step, the laser beam is directed at the distal end of the device to create a smooth surface. This final pass may use a larger ablation profile at a reduced power level compared to previous steps. The smoothing process aims to eliminate any remaining surface irregularities and create a uniform, atraumatic surface. The result of the smoothing step 108 may be referred to as the smoothed tip 400.

The method concludes after the smoothing step 108 is completed, resulting in an atraumatic tip that may reduce the risk of tissue damage during device navigation while maintaining the necessary functional characteristics of the device, such as energy transfer capabilities for ablation procedures.

This systematic approach, combining shaping, filleting, and smoothing steps, allows for precise control over the tip geometry and surface characteristics. The iterative nature of the process, particularly in the filleting stage, enables fine-tuning of the tip profile to meet specific design requirements for atraumatic performance and functional efficacy.

Each of these steps is discussed in more detail below.

In an embodiment, the puncture device comprises a body having a tip located at the distal end of the body. The tip may comprise an access means configured to puncture the desired region and permit the passage of any of the body through the puncture. For example, the access means may permit the tip to puncture the fossa of the heart and the body may be a cannula and/or catheter that permits access to the transseptal space in the heart. Of course, other regions and/or purposes are contemplated for use with the present disclosure.

The system may include a generator configured to produce and deliver energy to the distal tip of the device. This generator may be a radiofrequency (RF) electrical generator designed to operate in a high impedance range, which may be necessary due to the small size of the energy delivery tip. The generator may be capable of delivering energy as a continuous wave at frequencies between about 400 kHz and about 550 kHz, such as about 460 kHz, with a voltage between 100 to 200 V RMS and for durations up to 99 seconds. Alternatively, the generator may be configured to deliver pulsed or non-continuous RF energy, with parameters such as power output not exceeding about 60 watts, voltage ranging from about 200 Vrms to about 400 Vrms, and duty cycles between about 5% and 50% at frequencies from slightly above 0 Hz to about 10 Hz. The system may also include a grounding pad coupled to the generator to provide a return path for the RF energy when operated in a monopolar mode.

The energy delivery system may further comprise additional components to facilitate energy transmission and monitoring. An electrocardiogram (ECG) interface unit may function as a splitter, allowing simultaneous connection of the electrosurgical tissue piercing apparatus to both an ECG recorder and the generator. This setup may enable continuous monitoring and recording of ECG signals while energy is being delivered. The ECG interface unit may include a filtering circuit that permits energy delivery from the generator through the electrosurgical apparatus without compromising the ECG recording. Additionally, the system may include an adapter configured to releasably couple the apparatus to an external pressure transducer, which in turn may be connected to a monitoring system for converting and displaying pressure signals as a function of time. This configuration may allow for real-time monitoring of pressure at the distal tip during the procedure.

In some embodiments, the access means may be an atraumatic access means. In one embodiment, the access means may be configured as a distal electrode tip. The distal electrode tip may be an electrically conductive portion positioned on a distal end of the device configured to apply energy to the desired region to create the puncture. A person of ordinary skill will recognize various means for providing energy to the distal electrode tip to create a puncture.

The tubular body may comprise a distal end face that may define the distal electrode tip. The distal electrode tip may be configured to provide a surface to contact the target region to create the puncture. The delivery of energy through the distal electrode tip to create a puncture is known in the art and any manner of creating the puncture may be utilized.

The body may be a tubular body; however, other types of bodies are contemplated and may be utilized. For example, the body may be a solid body or a hollow body in any shape. Reference is made throughout to the tubular body, but any body may be utilized and should not be limited as such. In some embodiments, the tubular body may comprise a coating on any of an inner and/or outer surface of the tubular body. It is contemplated that the coating may be applied or otherwise adhered to any surface of the tubular body. The coating may be applied as part of the method or may be pre-existing on the tubular body as part of the method. In an embodiment, the tubular body may comprise a jacket on any of the inner and/or outer surface of the tubular body.

In some embodiments the coating may be an insulation layer around any surface or portion of the tubular body. In an embodiment, wherein the access means is the distal electrode tip, the insulation layer may prevent the application of energy on or through the insulated portion of the tubular body. Thus, the placement of the insulated portion on the tubular body may allow for localized or targeted application of energy. Of course, the coating may comprise additional properties, such as anticoagulation, antimicrobial, or surface friction reduction, and the like.

In an embodiment, the atraumatic tip may be formed using the laser to create a filleted dilation portion of the tip.

In one embodiment, a mandrel may be inserted into a channel defined by the inner surface of the tubular body prior to ablation. The mandrel may be operative to prevent the laser from contacting an inner surface of the tubular body. Of course, other methods for preventing contact with the inner surface of the tubular body may be utilized. Further, in some embodiments, the mandrel may be utilized to position the tubular body during the application of the laser to create the filleted dilation portion. For example, the mandrel may be configured to rotate the tubular body along a longitudinal axis. In another embodiment, another means, or device may be utilized to rotate the tubular body along the longitudinal axis.

In one embodiment, a system for creating the atraumatic tip may comprise the laser and a means for coupling to the device. Further, in some embodiments, the laser may be electronically coupled to a computer comprising computer-executable instructions operative to instruct the laser to perform the method.

An ablation profile may be a series of instructions provided to the laser configured to create a desired shape on the distal end of the tubular body. The ablation profile may be a computer-executable instruction configured to instruct the laser to perform a series of steps to create the desired shape. In one embodiment, the ablation profile may be a two-dimensional template that defines a path for the laser to follow to ablate the distal tip of the tubular body. The two-dimensional template may, for example, utilize rasterization to create the two-dimensional template for the laser.

The ablation profile may further comprise a power level of the laser. The power level of the laser may be determined according to the desired tip profile. For example, cutting the tip may comprise a higher power level than smoothing the tip and thus the power level may be varied according to the desired results.

In one embodiment, the tubular body may rotate about a longitudinal axis defined by a midpoint of the radius of the tubular body. In such an embodiment, the laser may move along a two-dimensional axis as the tubular body rotates along the longitudinal axis. However, in another embodiment, the laser may rotate about the tubular body.

A first tip profile may be created by the laser in a shaping step. The shaping step may comprise cutting the distal end of the tubular body into an initial shape. The first tip profile may be created by a laser beam emitting from the laser passing over the distal end of the tubular body according to a first ablation profile. The power level may be any power level suitable for cutting through the tubular body. In one embodiment, the power level may be designed to cut through the tubular body in multiple passes of the laser. For example, it may take about eight passes of the laser to cut through the tubular body. However, in other embodiments, more or less passes may be necessary to cut through the tubular body. Still, in further embodiments, the first tip profile may be formed separately from the laser beam and the tubular body provided to the laser may already comprise the first tip profile.

Further, in some embodiments, the shaping step may create a squared distal end face on the tubular body. It is contemplated that by creating the squared distal end face induces a uniform cut such that the distal end face has a consistent diameter throughout. Of course, in other embodiments, the shaping step may create a different shape on the distal end face of the tubular body. The initial shape formed by the shaping step may be a geometry conducive for a given medical application.

In one embodiment, creating the first tip profile may further comprise creating a non-orthogonal or bevel face at the distal end of the tubular body. Reference is made throughout to the bevel face; however, it should be interpreted broadly and may be used interchangeably with non-orthogonal.

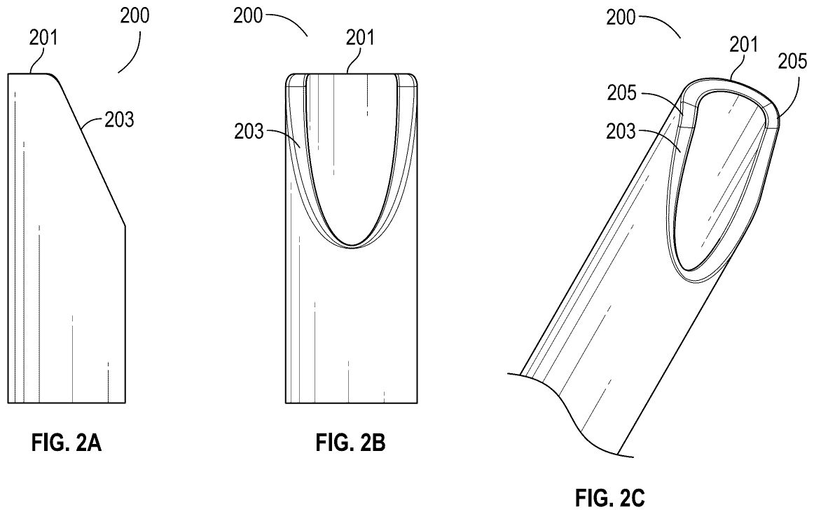

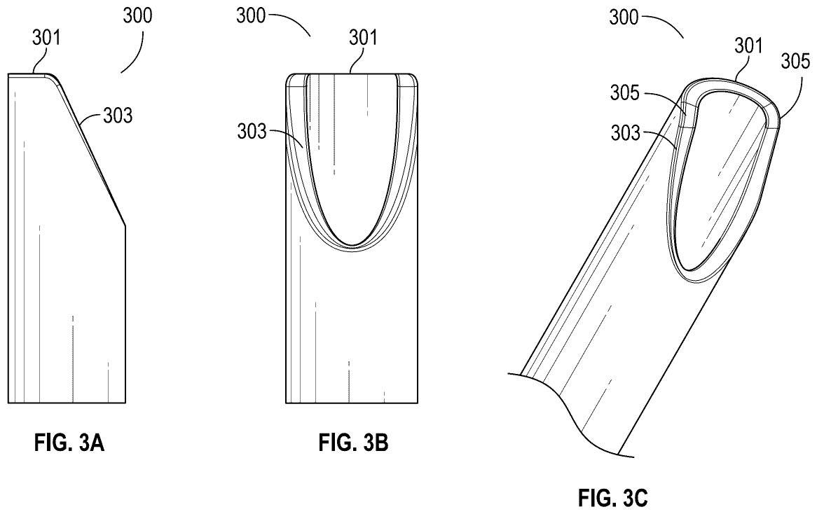

Returning to the embodiment comprising the bevel (e.g., as shown in FIGS. 2A-2C, 3A-C, and 4A-4G), the distal end of the tubular body may be defined by a first smoothed section 401 that is positioned generally perpendicular to a longitudinal axis of the tubular body and a second smoothed section 403 that is positioned at a non-orthogonal angle relative to the longitudinal axis of the tubular body. The first smoothed section 401 and second smoothed section 403 may, in some instances, improve the ability of the tubular body to puncture the desired region, with the leading first smoothed section 401 configured to penetrate the desired portion and the second smoothed section 403 configured to dilate the penetrated portion. This configuration may minimize the risk of coring in tissue penetration by creating a small tissue flap resulting in an expandable tissue aperture for receiving the device therethrough.

In one embodiment, the angle between the longitudinal axis of the tube and the second smoothed section 403 may be from about 10° to about 70°. In some instances, the angle may be between about 15° to about 50° or, more specifically, from about 20° to about 30°. In another embodiment, the angle may be about 25.37°.

The first smoothed section 401 and the second smoothed section 403 may comprise any size that may be desired. In one embodiment, the second section may comprise about 5% to about 75% of a diameter of the distal end face of the tubular body. In some embodiments, the second smoothed section 403 may comprise from about 10% to about 50%, or more specifically, about 25% to about 30% of the diameter of the distal end face. In one embodiment the first smoothed section 401 may be from about 20% to about 50% of the diameter of the distal end face of the tubular body.

FIGS. 2A-2C illustrates one embodiment of the distal end of the tubular body following the shaping step. For the purposes of this disclosure, the distal end of the tubular body shown in FIGS. 2A-2C and resulting from the shaping step may be referred to as the shaped tip 200. In many embodiments, the shaping step may produce a non-orthogonal or beveled face at the distal end of the device. This beveled profile may be characterized by two distinct sections: a first section shaped 201 that is positioned generally perpendicular to the longitudinal axis of the device, and a second shaped section 203 that is positioned at an angle relative to the longitudinal axis. The angle between these sections may vary depending on the specific requirements of the device, but it may typically range from about 100 to 140 degrees, with some implementations using an angle between about 110 to 130 degrees.

The creation of this beveled profile may serve several important functions. The first section, being perpendicular to the device axis (longitudinal axis), may act as the primary contact point with tissue. This flat face may provide a stable surface for energy delivery in applications such as RF ablation. The second, angled section may facilitate a gradual dilation of tissue as the device is advanced, potentially reducing the force required for insertion and minimizing trauma to surrounding tissues.

The relative proportions of these sections may also be carefully controlled. For example, the first section may comprise about 5% to about 75% of the diameter of the distal end face, with some embodiments utilizing a range of about 25% to about 30%. This configuration may help balance the device's ability to penetrate tissue effectively while maintaining an atraumatic profile.

In some applications, such as transseptal puncture devices, this beveled shape may be particularly advantageous. The leading edge formed by the first section 201 may be designed to create an initial puncture, while the angled second section 203 may gently dilate the opening. This may help minimize the risk of coring or tearing tissue, instead creating a small tissue flap that results in an expandable aperture for the device to pass through.

An initial coated tube may be cut to form the shaped tip 200, which comprises a first shaped section 201, a second shaped section 203, and shaped inflection sections 205. The first shaped section 201 may be positioned at the most distal end of the shaped tip 200 and may be flat or relatively flat, lying in a first plane that is orthogonal to the tube axis. The second shaped section 203 may be disposed at an angle relative to the first shaped section 201, where this angle may be approximately 110 degrees, though it may range from 100 to 140 degrees. The shaped inflection section 205 may be sufficiently rounded to avoid potential damage during use of the device. The first shaped section 201 may account for approximately 20-40 percent of the circumference of the shaped tip 200, though in another embodiment, it may account for approximately 50 percent of the circumference. This configuration may create a tip profile that balances the need for a flat surface for energy delivery with an angled section to facilitate easier crossing of tissue when energy is applied, while maintaining an atraumatic profile. The shaped tip 200 profile is configured to be later filleted, providing an initial profile where a laser can more carefully be utilized to fillet the tip, allowing for precise control over the final geometry and surface characteristics of the atraumatic tip.

The precise control over this initial shape is made possible by the use of laser cutting technology. The laser's ability to make highly accurate cuts with minimal thermal impact on surrounding material (e.g., insulating layers) may allow for the creation of complex geometries that would be difficult or impossible to achieve with traditional machining methods.

This carefully shaped profile serves as the starting point for the subsequent filleting and smoothing steps. By beginning with a well-defined geometry, the later stages of the process can more effectively refine the tip into its final atraumatic form while preserving the functional characteristics necessary for its intended medical application.

Next, the laser beam may be applied to the first tip profile to create a fillet on the distal end face of the tubular body. In some embodiments, the laser beam may be applied at various levels and passes in order to create the fillet on the distal end face. For example, various tip profiles may be utilized, wherein each tip profile defines comprises the ablation profile and power level. Each of these tip profiles may build on each other to create the fillet on the distal end face.

For example, a second tip profile may be created by the laser beam on the first tip profile to form the fillet. The laser beam may pass over the tip with a 200 μm diameter ablation profile. The number of passes necessary to create the second tip profile may be any number of passes. In one embodiment, the number of passes may be three passes, however, more or less passes may be utilized.

For the purposes of this disclosure, the diameter ablation profiles referenced in the method (e.g., 200 μm, 100 μm, 50 μm) describe a circular window within which the laser beam is rastered, or very quickly swept back and forth, during each pass. This circular window defines the area over which the laser energy is distributed during a single pass. As the diameter of this window increases, the effective laser power is reduced proportionally to the area within the circle. This occurs because the same laser beam must traverse more distance to ablate the same area when the diameter is larger. The sequence of passes with different sweep diameters and relative intensities is designed to create a fillet with a “stair step” profile initially. The final smoothing pass, which uses a large diameter and lower power, serves to blend the edges of these “stair steps” created by the previous passes. This approach allows for the creation of an approximate radial profile when viewed in cross-section. The specific diameters used for each pass are carefully tuned to achieve the desired final geometry of the atraumatic tip. While it may not be straightforward to point to these diameter measurements directly on a drawing of the finished tip, they are crucial parameters in the manufacturing process that determine the final shape and surface characteristics of the atraumatic tip.

Continuing with the example, a third tip profile may be created by the laser beam on the second tip profile to further form the fillet. The third tip profile may be created by the laser beam passing over the second tip profile with a 100 μm diameter ablation profile. Any number of passes may be utilized to form the third tip profile, including, for example, four passes.

A fourth tip profile may be created by the laser beam on the third tip profile to further form the fillet. The fourth tip profile may be created by the laser passing over the third tip profile with a 50 μm diameter ablation profile. The laser beam may pass over the third tip profile any number of times to create the fourth tip profile, including, without limitation, four passes.

While the second, third, and fourth tip profiles are described with specific diameter ablation profiles, any diameter ablation profile may be utilized and the aforementioned are solely provided as non-limiting examples. Indeed, the diameter of the ablation profile may be determined according to a desired fillet and/or number of tip profiles. As such, the filleting step may continue until the distal end has been sufficiently filleted as determined in the ablation profile. In one embodiment, the distal end may be sufficiently filleted when the surface area of the distal electrode tip is minimized while creating an atraumatic tip. For example, the distal electrode tip may comprise a uniform radius, such as the distal electrode tip shown in FIGS. 3A-3C.

The fillet produced by this step may be characterized by a curved transition between the different sections of the tip profile. For example, in a device with a beveled tip, the fillet may create a gradual arc that connects the perpendicular first section with the angled second section, eliminating any sharp corners or abrupt transitions.

The importance of this filleting process may be multifaceted:
1. Tissue protection: The rounded edges created by the fillet may significantly reduce the risk of tissue damage during device insertion and manipulation. Sharp edges can potentially cut or tear delicate tissues, while filleted edges may glide more smoothly through anatomical structures.
2. Improved navigation: A filleted tip may facilitate easier navigation through complex anatomical pathways. The smooth transitions may reduce catching or snagging on tissue structures, potentially allowing for more precise control and positioning of the device.
3. Stress distribution: From a mechanical perspective, filleted edges may help distribute stresses more evenly across the tip of the device. This may potentially increase the durability of the tip and reduce the risk of material failure during use.
4. Enhanced fluid dynamics: In applications where fluid flow around the device is important, such as in vascular procedures, a filleted tip may promote more laminar flow, potentially reducing turbulence and associated complications.
5. Optimized energy delivery: For devices used in energy-based treatments, the fillet may help create a more uniform surface for energy transfer. This may result in more predictable and controlled energy delivery to target tissues.

The filleting process may involve multiple passes with progressively smaller ablation profiles. For example, it may start with a 200 μm diameter profile, followed by 100 μm and 50 μm profiles. This gradual reduction in ablation profile size may allow for increasingly fine control over the fillet geometry, enabling the creation of smooth, consistent curves. The primary purpose of the filleting process in creating an atraumatic tip is to ensure that the device is not sharp on its own. This is a critical feature that distinguishes it from traditional sharp needles, such as the BRK needle, which are known to be difficult to control and may potentially puncture the fossa ovalis in an uncontrolled manner. The sharp nature of these traditional needles can lead to unexpected penetration, potentially causing the needle to jump through the fossa and damage the atrial wall in the left atrium. By creating a filleted profile, the device becomes inherently less likely to puncture tissue without the application of RF energy.

The filleted, non-sharp profile of the cannula provides a significant advantage in terms of procedural control and safety. It makes it difficult to puncture the fossa ovalis without the deliberate use of RF energy, ensuring that the cut or puncture only occurs when the physician intends it to happen. This controlled approach to tissue penetration may significantly reduce the risk of accidental damage to cardiac structures. Furthermore, when RF energy is applied, it takes less force to create the puncture, which may minimize the risk of damage to structures in the left atrium. This combination of a non-sharp profile and controlled RF energy application may allow for more precise and safer transseptal procedures, potentially improving patient outcomes.

The extent of the fillet may be carefully controlled to balance the atraumatic properties with the functional requirements of the device. For instance, in a transseptal puncture device, the fillet may need to be substantial enough to prevent tissue trauma during navigation, but not so extensive that it compromises the device's ability to create an initial puncture.

By creating a smooth, continuous surface that transitions gently between different sections of the tip, the filleting step may play a crucial role in enhancing both the safety and efficacy of the device. It may transform the initially sharp, potentially traumatic edges into a form that is more compatible with delicate biological tissues, while still maintaining the essential functional geometry established in the shaping step.

Following creating the fillet on the distal end face, the distal end face may be smoothed. While the aforementioned examples utilize three transitory tip profiles to generate the fillet, any number of transitory tip profiles may be utilized. It is contemplated that by smoothing the distal end face of the tubular body, any rough edges, lips, or other inconsistencies may be removed to create a smooth surface. The smooth surface is contemplated to reduce catching when puncturing and dilating the desired region, providing a smooth and atraumatic advancement of the tubular body. Continuing with the previous embodiment, a fifth tip profile may be created by the laser beam on the fourth tip profile. The fifth tip profile may be created by the laser beam passing over the fourth tip profile with a 300 μm diameter ablation profile. The laser beam may pass over the fourth tip profile any number of times required to create the smooth surface. For example, in some embodiments, the smoothing may be done in one pass. Of course, any number of passes may be utilized, and the diameter of the ablation profile may be any diameter suitable to create the smooth surface.

FIGS. 3A-3C illustrate one embodiment of the distal end of the tubular device following the filleting step. For the purposes of this disclosure, the distal end of the tubular body shown in FIGS. 3A-3C and resulting from the filleting step may be referred to as the filleted tip 300. As illustrated in FIGS. 3A-3C, the distal end face of the tubular device may comprise a first filleted section 301 and a second filleted section 301. The first filleted section 301 may be a leading first end face of the tubular body configured to penetrate tissue. The second filleted section 303 may be an inclined portion of the distal end of the tubular body extending proximally from the first section. The inclined portion of the distal end of the tubular body is contemplated to dilate the tissue that was penetrated by the leading first face of the tubular body.

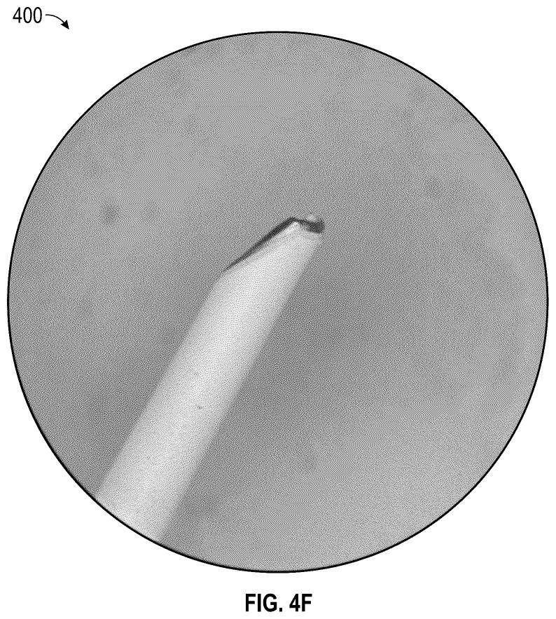

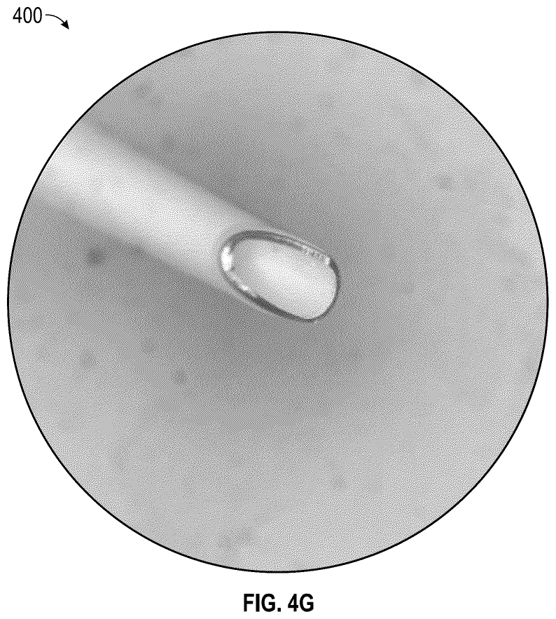

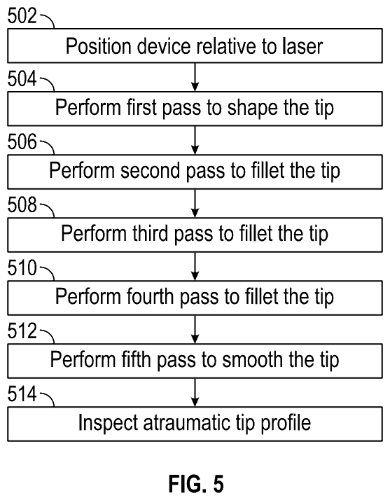

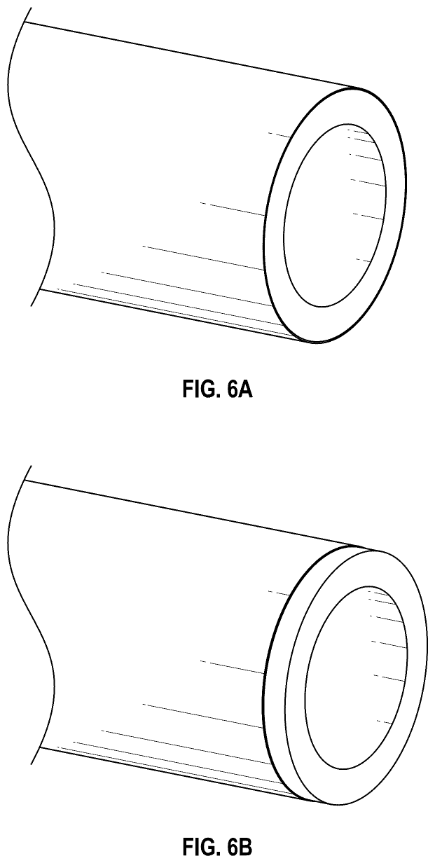

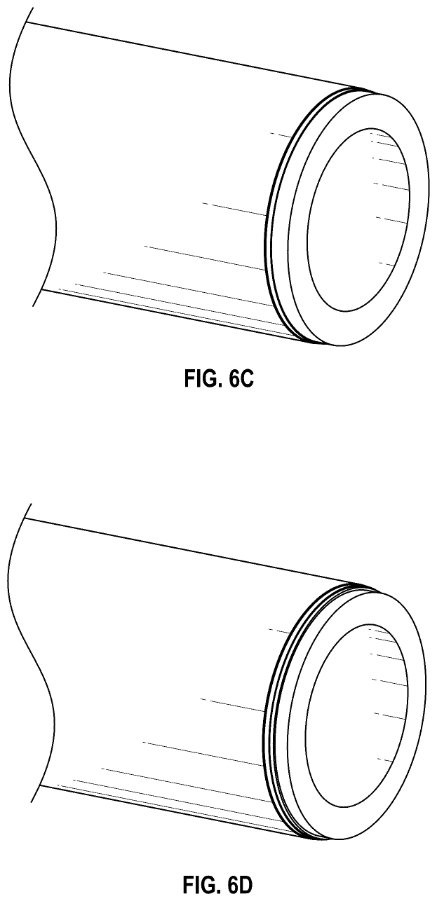

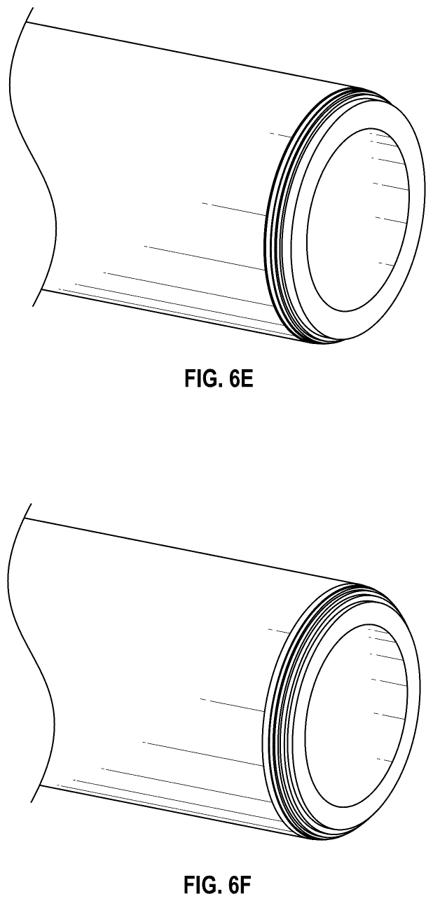

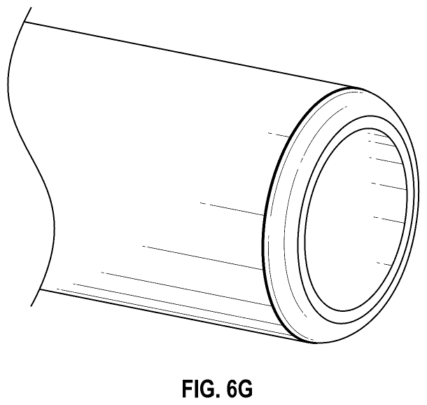

The shaped tip 200 may be converted to the filleted tip 300 through a series of laser ablation steps, resulting in a profile comprising a first filleted section 301, a second filleted section 303, and a filleted inflection section 305. The first filleted section 301 may lie in the first plane, which is orthogonal to the tube axis (longitudinal axis), while the second filleted section 303 may be disposed at an angle relative to the first filleted section 301. This angle may be approximately 110 degrees, though it may range from 100 to 140 degrees. The filleted inflection points 305 may be sufficiently rounded to prevent potential damage during device use. The first filleted section 301 may account for approximately 20-40 percent of the circumference of the filleted tip 300, or in another embodiment, it may account for approximately 50 percent. The filleting process may be more apparent in the embodiment shown in FIGS. 6B-6F, as the representations of the fillets are more visible when shown relative to a completely cylindrical distal tip, more clearly demonstrating the stepwise cuts made to the distal end of the tip. Accordingly, the filleted tip 300 may incorporate the fillets demonstrated in FIGS. 6B-6F, wherein said fillets are created relative to the shaped tip 200 profile having the first and second portions disposed at an angle from one another. This filleting process may result in a tip ready for the smoothing process.

The filleting process may create a series of stepwise edges in the tip relative to the outer wall of the tube. These stepwise edges may originate on the outer edge of the top surface of the front face of the tip and chamfer proximally along the outer surface of the tip and tube. This process may result in a rough angled area that transitions from the distal end of the tip towards the proximal direction of the tube, with each laser pass removing material in a controlled manner to create distinct steps or levels. The stepwise nature of these edges may provide a gradual transition from the tip to the outer surface of the tube, effectively distributing any forces encountered during use over a larger surface area. This stepped profile may serve as an intermediate stage in the creation of the final smoothed surface.

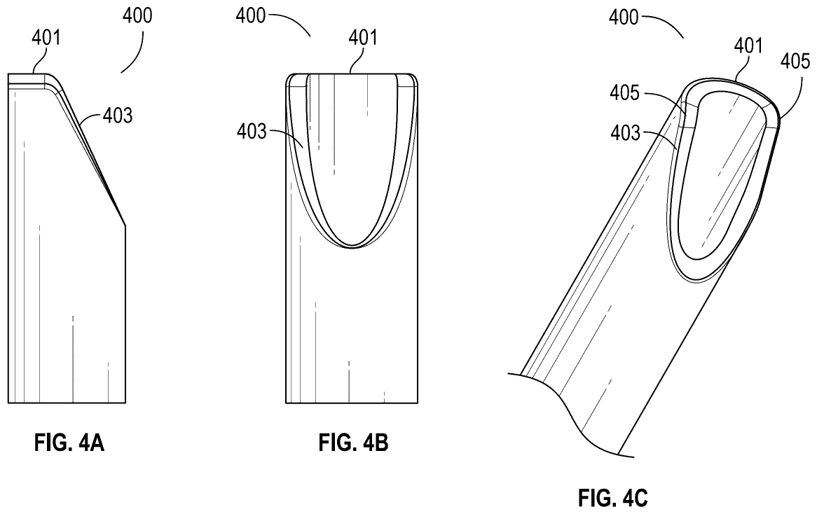

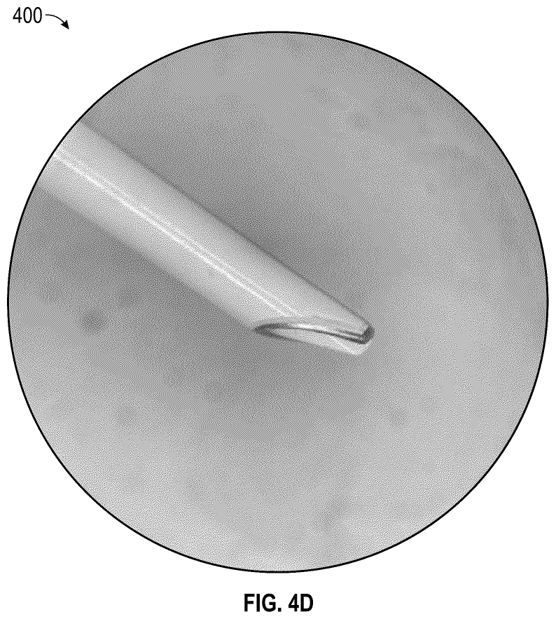

FIGS. 4A-4G show one embodiment of the distal end of the tubular device following the smoothing step. For the purposes of this disclosure, the distal end of the tubular body shown in FIGS. 4A-4G and resulting from the smoothing step may be referred to as the smoothed tip 400. As visible in FIGS. 4A-4G, the coating may be removed from the distal end face and may be filleted proximally to a full thickness of the coating. Smoothing the distal end of the tubular device is contemplated to provide a smooth transition between the electrode tip and the coating.

The filleted tip 300 may be converted to the smoothed tip 400 through a final laser ablation process, resulting in a profile comprising a first smoothed section 401, a second smoothed section 403, and smoothed inflection sections 405. The first smoothed section 401 may lie in the first plane, which is orthogonal to the tube axis, while the second smoothed section 403 may be disposed at an angle relative to the first smoothed section 401. This angle may be approximately 110 degrees, though it may range from 100 to 140 degrees. The smoothed inflection points 405 may be sufficiently rounded to prevent potential damage during device use. The first smoothed section 401 may account for approximately 20-40 percent of the circumference of the smoothed tip 400, or in another embodiment, it may account for approximately 50 percent. The smoothing process may feather the fillets created during the filleting process, effectively removing any remaining jagged edges. This feathering may result in a seamless transition between the different sections of the tip, creating a uniformly smooth surface that maintains the overall geometry established in the previous steps while eliminating any potential catch points or sharp edges that could cause tissue damage during use.

In some embodiments, the laser beam may be utilized at a reduced power when smoothing the fillet. For example, when cutting and filleting the distal end face the laser may use a higher power than when smoothing the distal end face. In one embodiment, when smoothing the distal end face the laser may be at about 75% power. However, in other embodiments, the laser may be from about 10% to about 90% power when smoothing.

In some embodiments, in creating the atraumatic tip, about half of the coating at the distal end of the body may be ablated, or otherwise smoothed, to create a smooth surface. Of course, any portion of the coating may be ablated, or otherwise smoothed, to create the smooth surface. In an embodiment, the coating is approximately 0.003″ thick. 0.0015″ may be removed on the final laser smoothing pass so that the as-cut edge of the paint does not leave a sharp corner. An axial length of approximately 0.010″ of coating may be ablated to half thickness all the way around the bevel and fillet.

The smoothing step in the atraumatic tip creation process may be critical for refining the overall surface quality and achieving the final desired profile. This step typically involves a final laser pass that focuses on creating a uniform, polished surface across the entire tip. The smoothing pass may remove the “stair step” shape from the previous passes, and may ensure that the entire cut face is smooth.

The smooth profile produced by this step may be characterized by:
1. Uniform surface texture: The smoothing process may eliminate any remaining micro-irregularities or roughness left from previous steps, resulting in a consistently smooth surface across the entire tip.
2. Gradual transitions: Any remaining subtle edges or transitions between different sections of the tip may be further softened, creating a more continuous and gentle profile.
3. Controlled material removal: The smoothing step may involve carefully controlled ablation of the device's coating or outer layer, potentially reducing the thickness in a uniform manner. The smoothing process achieves a delicate balance between removing and maintaining the device's coating to optimize both functionality and safety. This process may create a transition zone from an area with no coating (the electrode face) to an area with full coating thickness (approximately 0.003″ thick) over a short distance. This carefully controlled transition serves two crucial purposes. First, it creates a small surface area annular electrode for RF energy transfer, ensuring efficient and localized energy delivery. Second, it provides a smooth transition from the electrode face to the tube body. This smooth transition is essential for preventing skiving and tissue damage, as well as avoiding unwanted mechanical punctures. By carefully managing this coating transition, the device maintains its insulating properties where needed while also ensuring an atraumatic profile that minimizes the risk of unintended tissue interaction or damage during use.
4. Rounded edges: Any residual sharp edges or corners may be further rounded, enhancing the overall atraumatic nature of the tip.
5. Minimized tissue irritation: A highly smooth surface may reduce friction and potential irritation when the device is in contact with or moving through tissue. This may be particularly important for devices intended for prolonged placement or frequent manipulation.
6. Consistent energy delivery: In devices designed for energy-based treatments, a smooth electrode surface may promote more uniform energy distribution, potentially improving treatment efficacy and reducing the risk of localized “hot spots” that could cause unintended tissue damage.
7. Reduced risk of particulate generation: A smooth surface may be less likely to generate particulates during use, which could be important for reducing the risk of emboli in vascular applications (e.g., risk of skiving of the soft plastic dilator material during advancement of the cannula).
8. Enhanced cleaning and sterilization: For reusable devices, a smooth surface may facilitate more effective cleaning and sterilization processes, potentially reducing the risk of infection transmission.

The smoothing step typically utilizes a larger ablation profile (e.g., 300 μm) at a reduced power level compared to previous steps. This configuration may allow for gentle, uniform material removal across the tip surface. The goal may be to ablate approximately half of the device's coating thickness, ensuring that any remaining coating edges are not sharp while maintaining the overall integrity of the coating.

By creating a highly refined, smooth surface, this final step may significantly contribute to the device's overall performance, safety, and efficacy in its intended medical application. The smooth profile may represent the culmination of the entire laser processing sequence, integrating the shaped geometry and filleted transitions into a cohesive, atraumatic tip design.

While the aforementioned embodiment is discussed having five tip profiles in the creation of the atraumatic tip, any number of tip profiles may be utilized to achieve a desired result. Each of the steps may comprise any number of tip profiles, including more or less than the aforementioned example. The total number of profiles may be determined according to any of the shape of the distal end face, the fillet, the desired dilation, overall width of the tubular body, or desired smoothness of the fillet.

In some embodiments, the intensity of the laser may be varied between each of the shaping, filleting, and smoothing steps. For example, the shaping step may utilize the highest intensity laser and the intensity of the laser may reduce in each subsequent step.

Further, in some embodiments, any of the steps may be combined with another of the steps.

Of course, while the aforementioned embodiments are discussed for an atraumatic puncture and the distal electrode tip, other access means may be utilized. For example, any portion of the tip may comprise a sharp face capable of piercing the desired surface. In such an embodiment, any of the tip profiles may be utilized to fillet the distal end of the tubular surface to improve dilation. It is contemplated that by filleting and smoothing the distal end, it may reduce catching of an undesired region.

It is contemplated that the process as described may be uniform around the access means of the device. For example, the distal electrode tip may be a consistent size throughout the entirety of the distal end face. The distal electrode tip having a consistent size is contemplated to provide for uniform application of electrical energy to create the puncture. This may reduce inadvertent burning or other damage that may occur because of non-uniform energy application.

The electrode tip formed by the laser cutting method may have a specific and carefully designed shape to optimize energy delivery and safety. In an embodiment, when viewed down the bore of the tube, the electrode appears purely annular, creating a consistent circular profile. In such an embodiment, from a side view, the electrode exhibits a bull-nosed shape, characterized by a flat section and a beveled (angled) section. This dual-section design may serve important functional purposes. As a nonlimiting example, the flat shape may limit the risk of mechanical puncture when energy is not applied, while the bevel may facilitate easier crossing of the cannula once energy is applied.

The cross-sectional profile of the electrode may maintain consistency along the entire swept bevel shape. This uniform profile may ensure that the electrode has a uniform temperature distribution when energized. Additionally, the design aims to limit energy leakage into the blood pool by maximizing the surface area in contact with the tissue. Despite the complex swept shape of the electrode, its thickness remains uniform at all locations. This uniformity in thickness and cross-sectional profile may contribute to the consistent size of the distal electrode tip throughout the entirety of the distal end face, potentially enabling uniform application of electrical energy for creating the puncture and reducing the risk of inadvertent burning or damage from non-uniform energy application.

The optimal profile for RF energy transfer may feature a smooth, uniform conductive surface with a carefully controlled geometry that balances energy delivery efficiency with atraumatic characteristics. This profile may include a flat or slightly convex electrode face to maximize contact area with target tissue, surrounded by gently rounded edges to prevent unwanted tissue damage during positioning. The method described herein may be particularly well-suited for producing this type of profile due to its precise, multi-step approach. The initial shaping pass creates the basic electrode geometry, while subsequent filleting passes gradually round the edges to reduce the risk of tissue trauma. The final smoothing pass may be crucial for creating a uniformly smooth electrode surface, which may enhance energy distribution and reduce the likelihood of localized hot spots during RF application. By utilizing progressively finer ablation profiles followed by a larger, lower-power smoothing pass, this method may allow for exquisite control over the final tip geometry and surface characteristics. The ability to fine-tune each step of the process may enable the creation of an electrode profile that optimizes RF energy transfer while maintaining an atraumatic design, a combination that may be challenging to achieve with traditional manufacturing methods. Furthermore, the laser-based approach may minimize thermal effects on the electrode material, potentially preserving its conductive properties and ensuring.

Further, it is contemplated that the process as described may be utilized with various geometries. For example, the process may be utilized for various bevel designs, sizes, surface areas, and to form non-uniform distal electrode tips.

### Example Embodiment 1

The tip profile cutting procedure may include a plurality of “passes,” wherein each pass or phase of passes includes a given ablation profile, angle, number of passes/rotations, and/or power level. The “passes” of Example Embodiment 1 below depict one implementation of the methodology described herein and are not intended to limit the potential embodiments or use cases of the present disclosure.
PASS 1 (shaping): Cut tip profile “square”—the ablation profile may be approximately 30 μm in diameter and power may be tuned such that it takes roughly 8 passes/rotations to cut through the tube.
PASS 2 (filleting): 200 μm diameter ablation profile, 3 passes/rotations.
PASS 3 (filleting): 100 μm diameter ablation profile, 4 passes/rotations.
PASS 4 (filleting): 50 μm diameter ablation profile, 4 passes/rotations.
PASS 5 (smoothing): Smoothing—300 μm diameter ablation profile, 1 pass/rotation, 75% power. The goal of pass 5, in this nonlimiting example, is to ablate half of the coating so that the coating edge is not sharp.

While reference is made throughout to the puncture device, it is contemplated that any device may be utilized without deviating from the scope of the invention. For example, the atraumatic tip may be created on a device configured for use with pulsed-field ablation or other purposes.

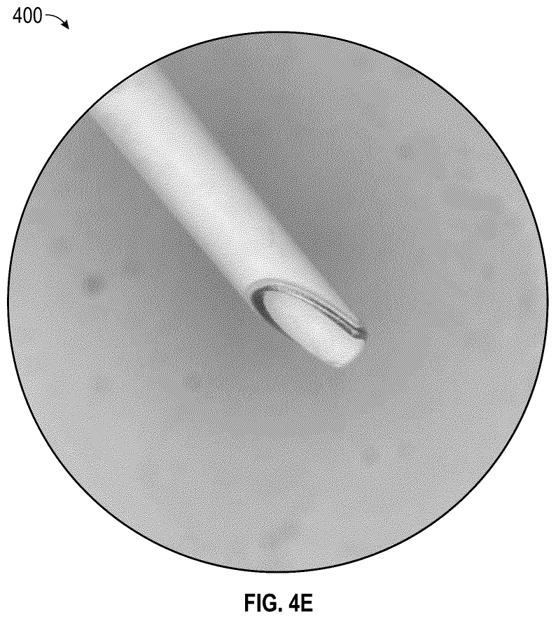

Referring to FIG. 5, in some embodiments, the method for creating an atraumatic tip may involve a series of laser passes, each with specific parameters to achieve the desired tip profile. The method may begin with step 502, positioning the device relative to the laser.

Referring to step 504, the first pass may utilize an ablation profile of approximately 30 μm in diameter. The power of the laser may be adjusted such that it takes about 8 passes or rotations to cut through the tube. In another embodiment, the number passes or rotations during this first pass may be 6-10 passes or rotations. This initial pass may create a squared profile on the distal end of the device, providing a foundation for subsequent shaping. The precise control of the laser power and number of passes may help ensure a clean, consistent cut without excessive thermal effects on the surrounding material.

Referring to step 506, a second pass may be performed using a 200 μm diameter ablation profile, involving 3 passes or rotations of the device relative to the laser beam. This larger ablation profile may begin to shape and refine the initially cut squared profile. The increased diameter may allow for more rapid material removal while maintaining control over the overall shape. This pass may start to create the general contour of the atraumatic tip.

Referring to step 508, following the second pass, a third pass may be conducted using a 100 μm diameter ablation profile, involving 4 passes or rotations. The reduced ablation profile diameter may allow for more precise shaping of the tip profile, further refining the geometry created in the previous passes. This step may be crucial in developing the smooth transitions and curves characteristic of an atraumatic tip.

Referring to step 510, a fourth pass may then be performed using a 50 μm diameter ablation profile, again involving 4 passes or rotations. The even smaller ablation profile may allow for fine detailing of the tip profile, creating smoother transitions and more precise geometries. This pass may be particularly important in eliminating any sharp edges or abrupt transitions that could potentially cause tissue trauma.

Referring to step 512, a fifth pass may be conducted using a 300 μm diameter ablation profile. This pass may involve a single pass or rotation at approximately 75% of the power used in previous passes. The goal of this smoothing pass may be to ablate about half of the coating on the device, ensuring that the coating edge is not sharp. This final pass may create a smooth, atraumatic surface on the tip of the device, potentially reducing the risk of tissue damage during insertion or manipulation.

Referring to step 514, the tip profile may be inspected to ensure the tip is sufficiently smooth.

The creation of a detailed, atraumatic tip profile may be crucial for minimizing tissue trauma during medical procedures. An atraumatic tip may reduce the risk of inadvertent tissue damage, perforation, or other complications that could arise from the use of devices with sharp or abrupt edges. The smooth, gradual transitions created by this multi-pass laser ablation process may allow the device to navigate through delicate tissues with minimal resistance or trauma. This may be particularly important in procedures involving sensitive structures or where precise control and minimal tissue disruption are essential. The carefully controlled laser passes may allow for the creation of complex geometries that would be difficult or impossible to achieve through traditional manufacturing methods, potentially enabling new device designs optimized for specific medical applications.

The specific dimensions and geometries of the tip created by this method may contribute significantly to its atraumatic properties and energy transfer capabilities. The progressive laser passes, starting with a 30 μm ablation profile and culminating in a 300 μm smoothing pass, may create a finely tuned tip geometry with smooth transitions. This may result in a tip with a radius of curvature that is large enough to prevent tissue trauma, yet small enough to maintain precise control during navigation. The beveled or non-orthogonal face of the tip, which may comprise between about 5% and about 75% of the distal end diameter, may provide a gradual transition that further enhances the atraumatic nature of the device. Simultaneously, the careful preservation of the electrode surface during the laser ablation process may maintain a consistent conductive area for energy transfer. This balance between the rounded, atraumatic profile and the preserved electrode face may allow for efficient energy delivery while minimizing the risk of unintended tissue damage during device advancement and positioning.

The method for creating an atraumatic tip may be executed on a computer-controlled laser cutting system, which may provide precise control over the laser parameters and device positioning throughout the process. The system may include a laser source, beam delivery optics, a motion control system for rotating and translating the device, and a computer interface for programming and controlling the entire process. The computer may be loaded with software that contains pre-programmed routines for each of the laser passes, including the specific ablation profile diameters, number of rotations, and power levels for each step.

The execution of the method may begin with the computer positioning the device relative to the laser beam using the motion control system. For each pass, the computer may then activate the laser with the appropriate power settings and control the rotation of the device to achieve the specified number of passes. The system may automatically adjust the laser focus and power output between passes to match the requirements of each step. Throughout the process, the computer may monitor key parameters such as laser power output, device rotation speed, and position to ensure consistency and accuracy. After completing all passes, the system may move the device to an inspection position or signal the operator that the process is complete. This computer-controlled approach may allow for high repeatability and precision in creating atraumatic tips across multiple devices.

The methods disclosed herein for creating an atraumatic tip may be encoded as computer-executable instructions and stored on a non-transitory computer-readable medium. These instructions may be configured to execute on a computer that is operatively coupled to a computer-controlled laser system. When executed, the instructions may cause the computer to control various aspects of the laser system, including laser power output, beam focusing, and device positioning mechanisms. The instructions may define a series of operations corresponding to each laser pass, specifying parameters such as ablation profile diameter, number of rotations, and power levels. The computer may also be programmed to adjust these parameters dynamically based on feedback from the laser system or predefined criteria. Additionally, the instructions may include routines for device alignment, process monitoring, and quality control checks. By storing the method as computer-executable instructions, the process may be easily replicated, modified, and optimized across different devices or production runs, potentially ensuring consistency and efficiency in the manufacturing of atraumatic tips.

FIGS. 6A-6G illustrate the progressive stages of creating a filleted edge on a coated tube using a laser ablation process. FIG. 6A shows the initial state of the native coated tube with a squared-off end. FIGS. 6B through 6F depict the results of successive laser passes, each contributing to the gradual formation of the fillet. With each pass, material is removed and the edge becomes increasingly rounded, transitioning from the initial sharp corner to a more curved profile. The early passes (FIGS. 6B and 6C) show the initial material removal and edge rounding, while the later passes (FIGS. 6D, 6E, and 6F) demonstrate the refinement of the fillet shape with smoother transitions and increased curvature. FIG. 6G illustrates the final result after a smoothing pass, showing a completed fillet with a uniform, smooth surface transition from the tube end to the side wall. This series of figures visually represents the step-by-step process of transforming a square-edged tube into one with an atraumatic, filleted profile through controlled laser ablation. FIGS. 6A-6G demonstrate the filleting step on a tube profile, as opposed to the beveled profile contemplated above. However, the progression of the filleting and smoothing steps shown in FIGS. 6A-6G may be replicated on a beveled profile.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “about” or “approximately.” For each such reference, it is to be understood that, in some embodiments, the value, feature, or characteristic may be specified without approximation. For example, where qualifiers such as “about,” “substantially,” and “generally” are used, these terms include within their scope the qualified words in the absence of their qualifiers. For example, where the term “substantially planar” is recited with respect to a feature, it is understood that in further embodiments, the feature can have a precisely planar orientation.

Any reference throughout this specification to “certain embodiments” or the like means that a particular feature, structure or characteristic described in connection with that embodiment is included in at least one embodiment. Thus, the quoted phrases, or variations thereof, as recited throughout this specification are not necessarily all referring to the same embodiment or embodiments.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 wt. %” is intended to mean “about 40 wt. %”.

A number of implementations have been described. Nevertheless, it will be understood that various modifications may be made without departing from the spirit and scope of the disclosure. Accordingly, other implementations are within the scope of the following claims.

1. A method for creating an atraumatic tip on a tubular device, comprising:
positioning a tubular device relative to a laser, the tubular device comprising a body and a distal end;
directing a laser beam to the distal end of the body in a shaping step to create a shaped tip, the shaping step comprising a first number of passes with a first ablation profile diameter;
directing the laser beam towards the shaped tip in a filleting step to create a filleted tip, the filleting step comprising a second number of passes with a second ablation profile diameter, wherein the second ablation profile diameter is larger than the first ablation profile diameter;
directing the laser beam to the filleted tip in a smoothing step to create a smoothed tip, the smoothing step comprising a third number of passes with a third ablation profile diameter, wherein the third ablation profile diameter is larger than the second ablation profile diameter;
wherein the smoothed tip comprises a first smoothed section positioned substantially perpendicular to a longitudinal axis of the tubular device and a second smoothed section positioned at a non-orthogonal angle relative to the longitudinal axis, and wherein the first smoothing section comprises between 20% to 50% of a distal end face of the tubular device.

2. The method of claim 1, wherein the first ablation profile diameter is approximately 30 μm.

3. The method of claim 2, wherein the second ablation profile diameter is approximately 200 μm.

4. The method of claim 3, wherein the third ablation profile diameter is approximately 300 μm.

5. The method of claim 1, wherein the first number of passes is greater than the second number of passes.

6. The method of claim 5, wherein the second number of passes is greater than the third number of passes.

7. The method of claim 1, wherein the filleting step further comprises a fourth number of passes with a fourth ablation profile diameter, wherein the fourth ablation profile diameter is smaller than the second ablation profile diameter.

8. The method of claim 7, wherein the fourth ablation profile diameter is approximately 100 μm.

9. The method of claim 7, wherein the filleting step further comprises a fifth number of passes with a fifth ablation profile diameter, wherein the fifth ablation profile diameter is smaller than the fourth ablation profile diameter.

10. The method of claim 9, wherein the fifth ablation profile diameter is approximately 50 μm.

11. The method of claim 1, wherein the smoothing step is performed at a lower power level than the shaping step and the filleting step.

12. The method of claim 11, wherein the power level of the smoothing step is approximately 75% of the power level used in the shaping step and the filleting step.

13. The method of claim 1, wherein the tubular device comprises a coating, and wherein the smoothing step ablates approximately half of the coating thickness at the distal end of the body.

14. The method of claim 1, wherein the non-orthogonal angle of the second smoothed section relative to the first smoothed section is between 100 degrees and 140 degrees.

15. The method of claim 1, wherein the smoothed tip comprises smoothed inflection sections between the first smoothed section and the second smoothed section.

16. A puncture device comprising:
a body having a distal end, the body formed from a cannula and a coating, wherein the coating is disposed coaxially externally to the cannula;
an atraumatic tip at the distal end of the body, the atraumatic tip comprising:
a first smoothed section positioned generally perpendicular to a longitudinal axis of the body;
a second smoothed section positioned at an obtuse angle relative to the first smoothed section; and
smoothed inflection sections between the first smoothed section and the second smoothed section, wherein the first smoothed section, the second smoothed section, and the smoothed inflection sections define a frontal annular portion of the distal end.

17. The puncture device of claim 16, the first smoothed section, the second smoothed section, and the smoothed inflection sections each incorporating both the cannula and the coating.

18. The puncture device of claim 17, wherein the cannula is electrically conductive and the coating is electrically insulative.

19. The puncture device of claim 18 further comprising a radiofrequency (RF) electrical generator configured to deliver RF energy to the distal tip.

20. The puncture device of claim 17, wherein the obtuse angle is between 100 and 140 degrees, and the first smoothed section accounts for 20-40 percent of the frontal annular portion of the distal end.
